(19) United States
(12) Patent Application Publication
(10) Pub. No.: US 2026/0021019 A1
(43) Pub. Date: Jan. 22, 2026

(54) MIXING VIAL

(71) Applicant: Balanced Pharma Incorporated, Cornelius, NC (US)

(72) Inventor: John Scott Keadle, Cornelius, NC (US)

(21) Appl. No.: 19/188,738

(22) Filed: Apr. 24, 2025

Related U.S. Application Data
(60) Provisional application No. 63/673,932, filed on Jul. 22, 2024.

(51) Int. Cl. A61J 1/20; A61C 19/08; A61J 1/22; A61M 5/315

(52) U.S. Cl. CPC A61J 1/2096; A61C 19/08; A61J 1/2041; A61J 1/22; A61M 5/31505

(57) ABSTRACT

Various implementations include a syringe system. The system includes a vessel, a stopper, and a dislodgeable plug. The vessel includes a hollow interior chamber. The stopper includes a hollow interior chamber with an open distal end. The dislodgeable plug has a body and a radially outwardly extending flange. The body has a longitudinal axis, a first axial end, and a second axial end. The dislodgeable plug is disposed within and sealing the open distal end of the stopper. The entire dislodgeable plug is fully dislodgeable from the open distal end of the stopper. The radially outwardly extending flange is disposed at the first axial end of the dislodgeable plug. The dislodgeable plug has two static resting positions consisting of a first static resting position in which the plug rests on the first axial end and a second static resting position in which the plug rests on the second axial end.

### BACKGROUND

For various reasons, some drugs must be stored and transported in two parts, and then mixed together just prior to administration. For example, steroid drugs in powder form are sometimes mixed with various vehicles just prior to injection and local anesthetics are sometimes mixed with NaHCO3 buffering solution just prior to injection.

In addition, some drugs require agitation immediately prior to injection, and therefore require headspace for effective agitation, while at the same time being susceptible to oxidative degradation which occurs during storage from the gas in the headspace. These drugs benefit from vials that eliminate headspace during storage, but allow headspace for agitation just prior to injection.

Local anesthetic solution exerts its effect by blocking transmission of the nerve signal in the area of the nerve where the solution has been deposited. Generally speaking anesthetic solutions must be acidified to an acid pH of about 4.0 (akin to vinegar) to obtain adequate shelf life. This acid solution is painful to the patient when injected, and is just one more reason that people don't like getting shots at the dentists' or physicians' office. In addition, the drug does not exert any effect at this low “storage” pH, so the patient must wait several minutes after injection before the numbness begins, while their body slowly brings the pH up to that of the body tissues, which is about 7.4.

Overwhelming evidence demonstrates that raising the pH of the anesthetic to a pH closer to neutral just prior to injection (“buffering”) greatly decreases the pain associated with injection, as well as the latency of the anesthetic effect. Because of this, many inventors over several decades have attempted to help make buffering a widespread practice in both dentistry and medicine, but at present no method has been satisfactory for a number of reasons.

Sodium bicarbonate (NaHCO3) in an 8.4% aqueous solution is the most common solution used to buffer anesthetic. The NaHCO3 solution and the local anesthetic solution are typically mixed in a ratio of about 9 parts anesthetic to 1 part NaHCO3 solution.

When physicians, such as dermatologists and plastic surgeons, buffer their anesthetic, they first draw anesthetic solution into their syringe from a vial, and then add NaHCO3 solution to the syringe by drawing it from a separate vial. The unused anesthetic and NaHCO3 solutions, in their respective vials, along with the needle and syringe, must be discarded after each patient. This protocol typically wastes more than 90% of the NaHCO3, and two vials are required. In addition, the mixing protocol is subject to human error and has resulted in protocol breaches, including improper drug ratios, sterility breaches, and intentional “re-use” of single-patient drug vials on other patients, with resulting adverse medical outcomes. This method is also somewhat cumbersome, time-consuming, and uneconomical. However, despite the risk and the cost in time and money, many physicians buffer their anesthetic, as it makes a significant difference in the patient's comfort when performed properly.

It would be preferable to have a system for the physician to draw and mix anesthetic medications using only one vial. Drawing from one vial instead of two would save valuable time, make the process safer, save the costs of the extra vial and the excess buffer solution, result in more widespread use of buffering, and increase patient comfort.

In the oral environment, because of the special need for dexterity and the likelihood of multiple injections on the same patient, dentists typically inject the anesthetic solution with a reusable hand-held metal syringe, which has evolved and consolidated into a single design that accounts for almost all syringes in use in dentistry and oral surgery. This syringe is used in conjunction with a disposable hollow injection needle which is attached to the syringe and a specialized glass drug vial called a “cartridge” or “carpule” of anesthetic solution, which is placed into the syringe. As the cartridge is placed into the syringe, the cartridge is pierced by the back end of the needle, so that solution can flow out and through the needle. The cartridge has a piston that is driven by a pushrod on the syringe, so that when the dentist pushes on the pushrod the piston drives the solution through the attached needle.

The cartridge has the shape of an elongated barrel. The cartridge is sealed on one end with a puck shaped elastomeric membrane that is fastened with a crimped metal cap in the manner typical of most glass drug vials A notable and important difference between a dental cartridge and a typical drug vial is the size. A dental cartridge volume is typically less than 2 ml, due mainly to limitations imposed by the syringe fitting in the operator's hand and its use in the mouth, whereas a typical drug vial has a volume of 10-100 ml.

Dentists who buffer their anesthetic typically use one of two commercially available systems. Both of these systems add considerable time and expense to the procedure. The ongoing product cost per injection of the commercially available systems is many times that of an unbuffered protocol. One system for dentists requires dentists to use a syringe/needle combination that is very different than the standard system that is ubiquitous in the industry. This system also requires a countertop device that contains both anesthetic solution and NaHC03 solution in separate vials and is used to load the disposable syringe/needle combination. This is time consuming, expensive, and its use is subject to operator error and breaches of sterile and pharmacological protocol.

Another system for dentists utilizes an injector that introduces NaHC03 into the standard dental cartridge. This system requires that each cartridge be separately loaded with NaHC03, which is time consuming, and in some states legally prohibited except by licensed pharmacists. In addition, this system causes a significant loss of available drug volume in the cartridge as well as being subject to operator error and breaches of sterile and pharmacological protocol.

In spite of their disadvantages, both systems have found a limited market, demonstrating that buffering is very desirable from both a dentist's and a dental patient's perspective. Making the buffering process less time consuming, less expensive, safer, and more similar to current surgical protocol would result in more widespread use for the benefit of both patient and dentist.

In the case of steroid drugs, physicians typically are provided with a special glass vial that has been partitioned into two chambers. The glass vial is provided with a plunger mechanism that the physician manually depresses to mix the contents of the chambers. The “Act-0-Vial” is typical of these special vials. The Act-0-Vial consists of seven parts and requires a specially shaped glass vial and apparatus.

The Act-0-Vial and similar special vials are also used in conjunction with drugs which require headspace for agitation, but benefit from a lack of headspace during storage. In this case, the fluid in the first chamber is stored without headspace, and then utilizes the headspace in the second chamber to permit agitation.

There remains a need to provide a less time consuming and less expensive method for in situ mixing or agitation of drugs, notably including the buffering of local anesthetic and the mixing of steroids for injection. Accordingly it is desired to provide various embodiments of the present invention as follows: to provide a mixing cartridge suited for use with dental syringes which may be manufactured more simply, using an extruded hollow glass tube that is cut to length, rather than a complicated form requiring each cartridge to be individually blown around a mandrel; to simplify the filling and sealing of the dental cartridge by requiring fewer and simpler parts; to eliminate the use of metal crimped caps for sealing, along with the metal dirt and dust which contaminates the clean or sterile assembly lines required for the manufacture of dental cartridges; to provide maximal interior volume to contain a drug; to facilitate the ability to eliminate gaseous headspace and the associated oxidative degradation of the drug in a dental cartridge; to provide a mixing dental cartridge that allows maximum interior volume for drug storage while minimizing breakage; to provide mixing or agitation of drugs used in medicine or dentistry using a minimum of simple parts; to reduce the possibility of operator error and breaches in sterile or pharmacological protocol when mixing drugs; to provide a mixing vial stopper which may be used with the typical medical vial and the typical crimped sealing cap, rather than requiring a specially shaped vial that is difficult to manufacture or a multipart sealing mechanism; to save time and cost, to facilitate accurate proportioning of materials to be mixed and avoid operator error; to make widespread the use of local anesthetic buffering; to reduce pain and waiting time in the administration of local anesthesia in medical and dental offices.

Other advantages of one or more aspects will be apparent from the following disclosure and claims taken in conjunction with the accompanying drawings.

### SUMMARY

Various implementations include a syringe system. The system includes a vessel, a stopper, and a dislodgeable plug. The vessel includes a hollow interior chamber having a proximal end and a distal end, and a neck adjacent the proximal end. The neck has a distal end and a proximal open end. The stopper includes a hollow interior chamber with an open distal end and a closed proximal end. The dislodgeable plug has a body and a radially outwardly extending flange. The body has a longitudinal axis, a first axial end, and a second axial end opposite and spaced apart along the longitudinal axis from the first end. The dislodgeable plug is disposed within and sealing the open distal end of the stopper. The entire dislodgeable plug is fully dislodgeable as a single piece from the open distal end of the stopper. The radially outwardly extending flange is disposed at the first axial end of the dislodgeable plug. The dislodgeable plug has two static resting positions consisting of a first static resting position in which the plug rests on the first axial end and a second static resting position in which the plug rests on the second axial end.

In some implementations, the distal end of the vessel is a distal open end.

In some implementations, the stopper defines an inwardly facing annular groove that receives the radially outwardly extending flange of the dislodgeable plug. In some implementations, the stopper defines a radially inwardly extending flange. In some implementations, the radially inwardly extending flange defines the inwardly facing annular groove.

In some implementations, the interior chamber of the vessel includes an anesthetic, and the interior chamber of the stopper includes a buffering reagent.

In some implementations, the stopper includes an elastomeric plastic or rubber.

In some implementations, the second axial end of the dislodgeable plug includes a strike plate. In some implementations, the dislodgeable plug is disposed within the open distal end of the stopper such that the strike plate faces the hollow interior chamber of the stopper. In some implementations, the strike plate is configured to resist penetration by the harpoon when the harpoon is used to dislodge the dislodgeable plug.

In some implementations, a portion of the body of the dislodgeable plug between the second axial end and the radially outwardly extending flange is tapered from a largest cross-sectional area adjacent the radially outwardly extending flange to a smallest cross-sectional area adjacent the second axial end. In some implementations, the first axial end defines a plug opening extending from the first axial end toward the second axial end. In some implementations, side walls of the plug opening are tapered such that the plug opening has a larger diameter adjacent the first axial end than it does adjacent the second axial end. In some implementations, the taper of the side walls of the plug opening includes a constant taper portion and a varying taper portion. In some implementations, the varying taper portion is disposed adjacent the first axial end and the constant taper portion is disposed adjacent the second axial end.

In some implementations, the stopper includes a stopper-piston. In some implementations, the entire stopper-piston is slidably positioned in a sealing relationship with the neck. In some implementations, the entire stopper-piston is slidable from a first position to a second position. In some implementations, the entire stopper-piston is adjacent the proximal end of the vessel when in the first position. In some implementations, the entire stopper-piston is closer to the distal end of the vessel than to the proximal end of the vessel when in the second position.

Various other implementations include a syringe system. The system includes a syringe with an axially movable plunger having a distal end defining a shoulder. A harpoon extends axially from the distal end of the plunger. The shoulder has a larger diameter than a harpoon minimum diameter. The harpoon is static with respect to the plunger. The system also includes the syringe system described above. When the syringe system is disposed in the syringe and the plunger is axially moved, the harpoon penetrates the closed proximal end of the stopper-piston, contacts the dislodgeable plug, and fully dislodges the dislodgeable plug prior to the shoulder of the plunger contacting the closed proximal end of the stopper-piston.

Various other implementations include a syringe system. The system includes a vessel, a stopper, and a dislodgeable plug. The vessel includes a hollow interior chamber having a proximal end and a distal end, and a neck adjacent the proximal end. The neck has a distal end and a proximal open end. The stopper includes a hollow interior chamber with an open distal end and a closed proximal end. The dislodgeable plug has a body and a radially outwardly extending flange. The body has a longitudinal axis, a first axial end, and a second axial end opposite and spaced apart along the longitudinal axis from the first end. The dislodgeable plug is disposed within and sealing the open distal end of the stopper. The entire dislodgeable plug is fully dislodgeable as a single piece from the open distal end of the stopper. The radially outwardly extending flange is disposed at the first axial end of the dislodgeable plug. The first axial end defines a plug opening extending from the first axial end toward the second axial end.

In some implementations, the distal end of the vessel is a distal open end.

In some implementations, the stopper defines an inwardly facing annular groove that receives the radially outwardly extending flange of the dislodgeable plug. In some implementations, the stopper defines a radially inwardly extending flange. In some implementations, the radially inwardly extending flange defines the inwardly facing annular groove.

In some implementations, the interior chamber of the vessel includes an anesthetic, and the interior chamber of the stopper includes a buffering reagent.

In some implementations, the stopper includes an elastomeric plastic or rubber.

In some implementations, the second axial end of the dislodgeable plug includes a strike plate. In some implementations, the dislodgeable plug is disposed within the open distal end of the stopper such that the strike plate faces the hollow interior chamber of the stopper. In some implementations, the strike plate is configured to resist penetration by the harpoon when the harpoon is used to dislodge the dislodgeable plug.

In some implementations, a portion of the body of the dislodgeable plug between the second axial end and the radially outwardly extending flange is tapered from a largest cross-sectional area adjacent the radially outwardly extending flange to a smallest cross-sectional area adjacent the second axial end.

In some implementations, side walls of the plug opening are tapered such that the plug opening has a larger diameter adjacent the first axial end than it does adjacent the second axial end. In some implementations, the taper of the side walls of the plug opening includes a constant taper portion and a varying taper portion. In some implementations, the varying taper portion is disposed adjacent the first axial end and the constant taper portion is disposed adjacent the second axial end.

In some implementations, the dislodgeable plug has two static resting positions consisting of a first static resting position in which the plug rests on the first axial end and a second static resting position in which the plug rests on the second axial end.

In some implementations, the stopper includes a stopper-piston. In some implementations, the entire stopper-piston is slidably positioned in a sealing relationship with the neck.

In some implementations, the entire stopper-piston is slidable from a first position to a second position. In some implementations, the entire stopper-piston is adjacent the proximal end of the vessel when in the first position. In some implementations, the entire stopper-piston is closer to the distal end of the vessel than to the proximal end of the vessel when in the second position.

Various other implementations include a syringe system. The system includes a syringe with an axially movable plunger having a distal end defining a shoulder. A harpoon extends axially from the distal end of the plunger. The shoulder has a larger diameter than a harpoon minimum diameter. The harpoon is static with respect to the plunger. The system also includes the syringe system described above. When the syringe system is disposed in the syringe and the plunger is axially moved, the harpoon penetrates the closed proximal end of the stopper-piston, contacts the dislodgeable plug, and fully dislodges the dislodgeable plug prior to the shoulder of the plunger contacting the closed proximal end of the stopper-piston.

Various other implementations include a mixing vial. The vial includes a vessel, a stopper, and a dislodgeable plug. The vessel includes a hollow interior chamber having a proximal end, a distal end, and a neck adjacent the proximal end. The neck has a distal end and a proximal open end. The stopper includes a hollow interior chamber with an open distal end and a closed proximal end. The dislodgeable plug has a body and a radially outwardly extending flange. The body has a longitudinal axis, a first axial end, and a second axial end opposite and spaced apart along the longitudinal axis from the first end. The dislodgeable plug is disposed within and sealing the open distal end of the stopper. The entire dislodgeable plug is fully dislodgeable as a single piece from the open distal end of the stopper. The first axial end includes a textured surface.

In some implementations, the second axial end of the dislodgeable plug includes a textured surface.

In some implementations, the textured surface defines two or more groove portions. In some implementations, the two or more groove portions are radially spaced apart from each other. In some implementations, the two or more groove portions extend parallel to a perimetrical edge of the first axial end. In some implementations, the two or more groove portions are arcuate or circular. In some implementations, the two or more groove portions have the same center point.

In some implementations, the textured surface defines a concave portion.

In some implementations, the distal end of the vessel is a distal open end.

In some implementations, the stopper defines an inwardly facing annular groove that receives the radially outwardly extending flange of the stopper. In some implementations, the stopper defines a radially inwardly extending flange, wherein the radially inwardly extending flange defines the inwardly facing annular groove.

In some implementations, the interior chamber of the vessel includes an anesthetic, and the interior chamber of the stopper includes a buffering reagent.

In some implementations, the stopper includes an elastomeric plastic or rubber.

In some implementations, the first axial end of the dislodgeable plug includes a strike plate. In some implementations, the dislodgeable plug is disposed within the open distal end of the stopper such that the strike plate faces the hollow interior chamber of the stopper. In some implementations, the strike plate is configured to resist penetration by the harpoon when the harpoon is used to dislodge the dislodgeable plug.

In some implementations, the radially outwardly extending flange is disposed between the first axial end and the second axial end of the dislodgeable plug.

In some implementations, a portion of the body of the dislodgeable plug between the first axial end and the radially outwardly extending flange is tapered from a largest cross-sectional area adjacent the radially outwardly extending flange to a smallest cross-sectional area adjacent the first axial end.

In some implementations, the stopper includes a stopper-piston. In some implementations, the entire stopper-piston is slidably positioned in a sealing relationship with the neck. in some implementations, the entire stopper-piston is slidable from a first position to a second position. In some implementations, the entire stopper-piston is adjacent the proximal end of the vessel when in the first position. In some implementations, the entire stopper-piston is closer to the distal end of the vessel than to the proximal end of the vessel when in the second position.

Various other implementations include a syringe system. The system includes a syringe and a mixing vial disclosed herein. The syringe has an axially movable plunger having a distal end defining a shoulder. A harpoon extends axially from the distal end of the plunger. The shoulder has a larger diameter than a harpoon minimum diameter. The harpoon is static with respect to the plunger. When the mixing vial is disposed in the syringe and the plunger is axially moved, the harpoon penetrates the closed proximal end of the stopper-piston, contacts the dislodgeable plug, and fully dislodges the dislodgeable plug prior to the shoulder of the plunger contacting the closed proximal end of the stopper-piston.

Various other implementations include a mixing vial. The vial includes a vessel, a stopper, and a dislodgeable plug. The vessel includes a hollow interior chamber having a proximal end, a distal end, and a neck adjacent the proximal end. The neck has a distal end and a proximal open end. The stopper includes a hollow interior chamber with an open distal end and a closed proximal end. The dislodgeable plug has a body and a radially outwardly extending flange. The body has a longitudinal axis, a first axial end, and a second axial end opposite and spaced apart along the longitudinal axis from the first end. The dislodgeable plug is disposed within and sealing the open distal end of the stopper. The entire dislodgeable plug is fully dislodgeable as a single piece from the open distal end of the stopper. A portion of the body of the dislodgeable plug between the first axial end and the radially outwardly extending flange is tapered from a largest cross-sectional area adjacent the radially outwardly extending flange to a smallest cross-sectional area adjacent the first axial end.

In some implementations, the first axial end includes a textured surface.

In some implementations, the second axial end of the dislodgeable plug includes a textured surface.

In some implementations, the textured surface defines two or more groove portions. In some implementations, the two or more groove portions are radially spaced apart from each other. In some implementations, the two or more groove portions extend parallel to a perimetrical edge of the first axial end. In some implementations, the two or more groove portions are arcuate or circular. In some implementations, the two or more groove portions have the same center point.

In some implementations, the textured surface defines a concave portion.

In some implementations, the distal end of the vessel is a distal open end.

In some implementations, the stopper defines an inwardly facing annular groove that receives the radially outwardly extending flange of the stopper. In some implementations, the stopper defines a radially inwardly extending flange. in some implementations, the radially inwardly extending flange defines the inwardly facing annular groove.

In some implementations, the interior chamber of the vessel includes an anesthetic. In some implementations, the interior chamber of the stopper includes a buffering reagent.

In some implementations, the stopper includes an elastomeric plastic or rubber.

In some implementations, the first axial end of the dislodgeable plug includes a strike plate. In some implementations, the dislodgeable plug is disposed within the open distal end of the stopper such that the strike plate faces the hollow interior chamber of the stopper. In some implementations, the strike plate is configured to resist penetration by the harpoon when the harpoon is used to dislodge the dislodgeable plug.

In some implementations, the radially outwardly extending flange is disposed between the first axial end and the second axial end of the dislodgeable plug.

In some implementations, the stopper includes a stopper-piston. In some implementations, the entire stopper-piston is slidably positioned in a sealing relationship with the neck. in some implementations, the entire stopper-piston is slidable from a first position to a second position. In some implementations, the entire stopper-piston is adjacent the proximal end of the vessel when in the first position. In some implementations, the entire stopper-piston is closer to the distal end of the vessel than to the proximal end of the vessel when in the second position.

Various other implementations include a syringe system. The system includes a syringe and a mixing vial as disclosed here. The syringe has an axially movable plunger having a distal end defining a shoulder. In some implementations, a harpoon extends axially from the distal end of the plunger. In some implementations, the shoulder has a larger diameter than a harpoon minimum diameter. In some implementations, the harpoon is static with respect to the plunger. In some implementations, when the mixing vial is disposed in the syringe and the plunger is axially moved, the harpoon penetrates the closed proximal end of the stopper-piston, contacts the dislodgeable plug, and fully dislodges the dislodgeable plug prior to the shoulder of the plunger contacting the closed proximal end of the stopper-piston.

### BRIEF DESCRIPTION OF DRAWINGS

Example features and implementations of the present disclosure are disclosed in the accompanying drawings. However, the present disclosure is not limited to the precise arrangements and instrumentalities shown. Similar elements in different implementations are designated using the same reference numerals.

FIG. 1 is a side elevation of a conventional needle/syringe apparatus for use in combination with a vial of the present invention.

FIG. 2 is a perspective view of a conventional vial having a single chamber.

FIG. 3 is an exploded, sectional view, in vertical section, of an embodiment of a mixing vial of the present invention with its dislodgeable plug in sealing position and a needle, broken away.

FIG. 4 is a sectional view, in vertical section, of an embodiment of FIG. 3 with its plug dislodged and a needle, shown broken away, the needle having been inserted into the vial to dislodge the plug.

FIG. 5 is a side elevation of a conventional dental cartridge needle/syringe apparatus for use in combination with a cartridge of the present invention.

FIG. 6 is an exploded sectional view, broken away, of the conventional dental cartridge needle/syringe apparatus of FIG. 5.

FIG. 7 is a perspective view of a conventional dental cartridge having a single chamber.

FIG. 8 is a sectional view of an embodiment of a mixing vial of the present invention in cartridge form with a needle inserted therein, the needle shown broken away.

FIG. 9A is a sectional view of an embodiment of FIG. 8, with a needle inserted therein, the needle shown broken away, after the harpoon-plunger of the syringe has been inserted into and through the hollow chambered piston and dislodged the plug.

FIG. 9B is a sectional view of an embodiment of FIG. 8, with a needle inserted therein, the needle shown broken away, and the blunt pushrod of the syringe has elongated the membrane of the stopper and dislodged the plug.

FIG. 10 is a sectional view, broken away, of an embodiment of a mixing vial of the present invention in cartridge form with a needle inserted therein, the needle having dislodged the plug to effect mixing of the two chambers' contents.

FIG. 11A is a sectional view of a disassembled mixing vial according to another implementation.

FIG. 11B is a sectional view of the mixing vial of FIG. 11A with the stopper disposed within the vessel.

FIG. 12A is an end view of the vessel of the mixing vial of FIG. 11A.

FIG. 12B is a side view of the vessel of the mixing vial of FIG. 11A with partial cutaway sectional views.

FIG. 12C is a detail sectional view of the proximal end of the vessel of the mixing vial of FIG. 11A.

FIG. 12D is a detail view of the distal end of the vessel of the mixing vial of FIG. 11A with partial cutaway sectional views.

FIG. 13A is a side view of the stopper of the mixing vial of FIG. 11A with partial cutaway sectional views.

FIG. 13B is a second end view of the stopper of the mixing vial of FIG. 11A.

FIG. 13C is a first end view of the stopper of the mixing vial of FIG. 11A.

FIG. 14 is a sectional view of a dislodgeable plug, according to another implementation, disposed within a stopper.

FIG. 15 is a perspective view of the dislodgeable plug of FIG. 14.

FIG. 16A is a top view of view of the dislodgeable plug of FIG. 14.

FIG. 16B is a side view of the dislodgeable plug of FIG. 14.

FIG. 17 is a cross-sectional view of the dislodgeable plug of FIG. 16B along lines 17-17.

FIG. 18 is a detail cross-sectional view 18-18 of the first axial end of dislodgeable plug of FIG. 17.

FIG. 19 is a sectional view of a dislodgeable plug, according to another implementation.

FIG. 20 is a top view of the dislodgeable plug shown in FIG. 19.

FIG. 21 is a sectional view of the dislodgeable plug shown in FIG. 20 along line 21-21.

### DETAILED DESCRIPTION

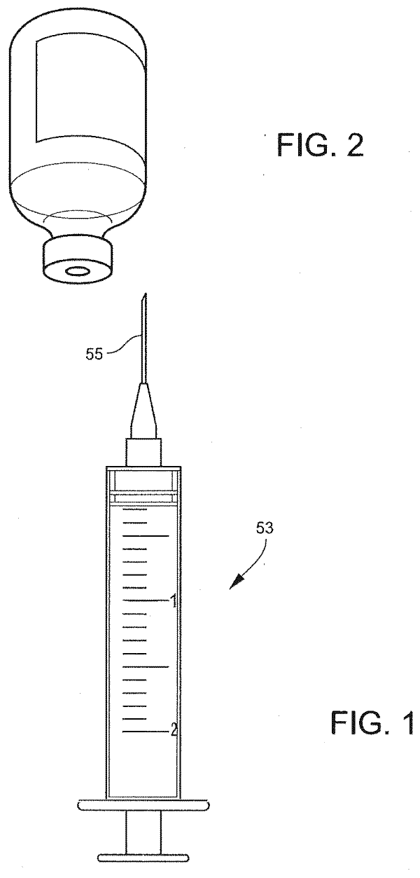

Now referring to the drawings, a conventional prior art syringe/needle apparatus is illustrated in FIG. 1 while a conventional prior art vial is illustrated in FIG. 2. The syringe/needle apparatus of FIG. 1 is suitable for use in conjunction with an embodiment of the mixing vial of the present invention as will be described in more detail in the following disclosure. The conventional prior art vial does not have dual chambers or a plug and is shown for illustrative purposes.

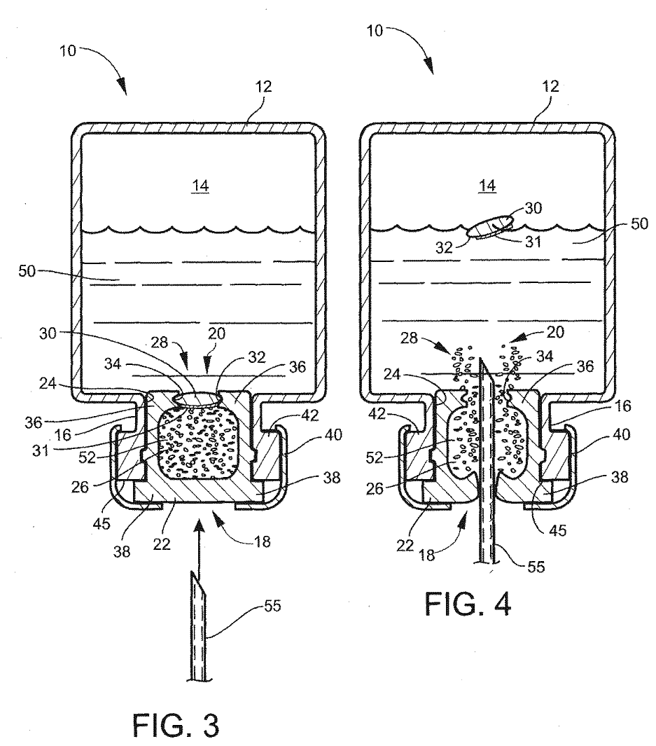

FIGS. 3 and 4 illustrate a mixing vial of the present invention which is indicated by the numeral 10. Mixing vial 10 provides the ability for physicians to buffer local anesthetic prior to injection in a manner that is economically feasible, is not excessively time-consuming, does not interfere with established surgical protocol, and does not require new or unfamiliar surgical equipment.

Broadly speaking, mixing vial 10 is a generally barrel shaped vessel 12 having a hollow interior chamber 14 and neck 16 having distal and proximal open ends, 18 and 20. An elastomeric, chambered stopper 22 is positioned in neck 16 and is in sealing relationship with interior facing wall 24 of neck 16.

Chambered stopper 22 has a hollow interior chamber 26 and an open proximal end 28 with a dislodgeable plug 30 positioned in and sealing said open end 28. Thus, annular edge 32 of plug 30 is tightly fit into annular groove 34 in radially inwardly extending stopper flange 36 of stopper 22. Vessel 12 may be made of glass, plastic or any other material suitable for use consistent with the purpose of the present invention.

Stopper 22 may be made of elastomeric plastic or rubber material, including the typical vial stopper material in present use, which will seal well with adjacent surfaces. Plug 30 may be made of PTFE, or any material or combination of materials suitable for sealing with stopper 22 and resisting penetration of the needle. For instance, plug 30 may have an elastomeric body and edge, with a PTFE strike plate 31 attached to the needle side to prevent needle penetration.

Stopper 22 has a radially outwardly extending annular flange 38 and is retained in position on neck 16 by metal clip 40 which clips onto annular shoulder 42 on neck 16 and compresses flange 38 against distal face 45 of neck 16.

As shown in FIGS. 3 and 4, interior chamber 14 of vessel 12 contains solution 50, for example, a local anesthetic solution, or a physiologic saline solution. Interior chamber 26 of stopper 22 contains a powder 52, for example, NaHCO3, or a powdered steroid medication.

In operation, the mixing vial 10 of the present invention is used in accordance with the following method. First, a mixing vial of the present invention is provided and its two chambers filled with suitable drugs or other materials which are desired to be kept separate and then mixed just before injection. Then, the vial is inverted and, using a typical disposable medical syringe 53 as illustrated in FIG. 1, the physician inserts the Syringe Needle 55 into the chambered stopper 22 as illustrated in FIGS. 3 and 4.

The physician must make certain that the Syringe Needle passes through chamber 26 of stopper 22 and dislodges plug 30 into the chamber 14 of mixing vial 10. The dislodged plug 30 may float to the top of the mixed solution, or alternatively be configured to stay in solution to aid in mixing the drugs. Also, vial 10 may be shaken to assist in mixing. Coloring or clouding reagents may be added to either chamber to provide visual indication of mixing or premature seal failure. The form of the Chambered Vial Stopper assembly allows elimination of headspace gas in one or the other of the chambers. The physician withdraws the mixed drug solution from the inverted vial into the syringe apparatus and administers it to the patient in the typical manner.

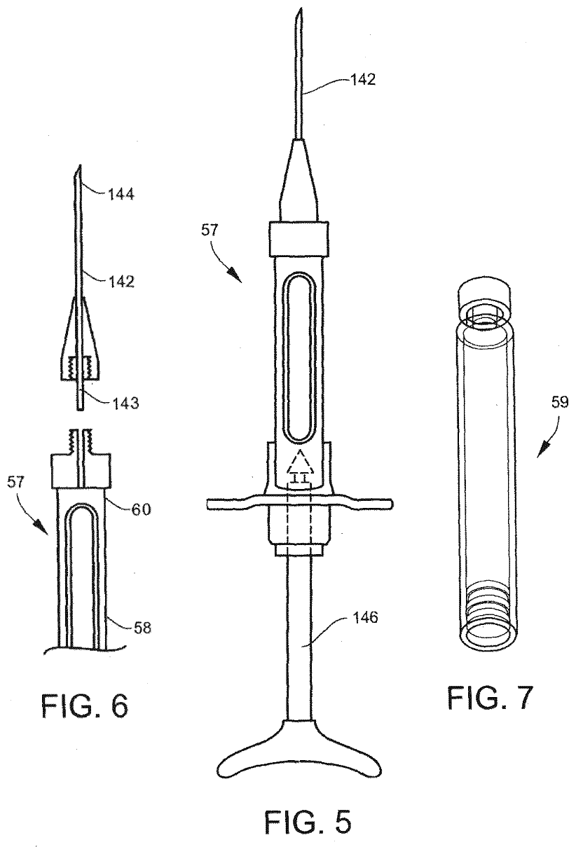

Now referring to FIGS. 5 and 6 a conventional syringe 57 for use with vials configured as cartridges such as the conventional dental cartridge 59 shown in FIG. 7 is illustrated. The conventional cartridge syringe is also useful for use with a cartridge style mixing vial of the present invention as further described below.

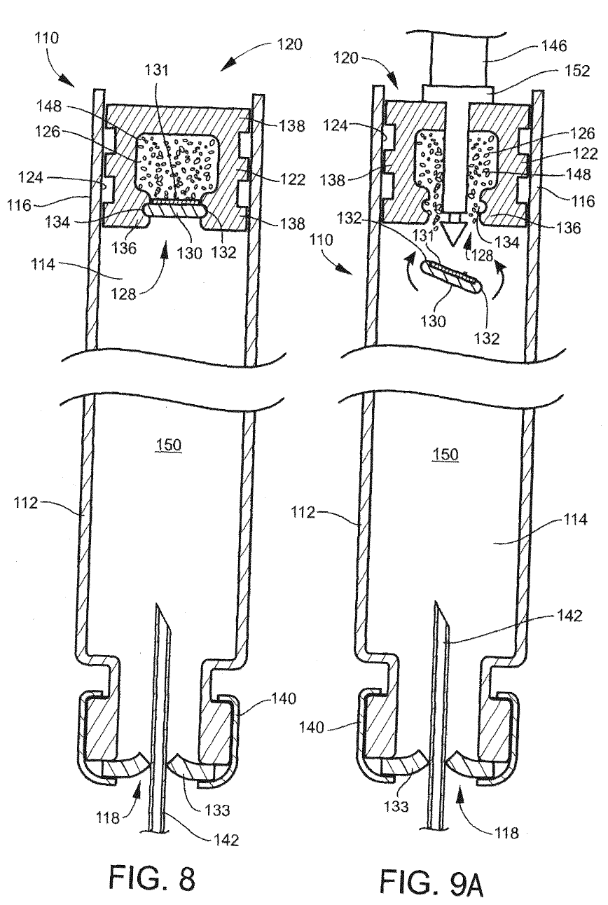

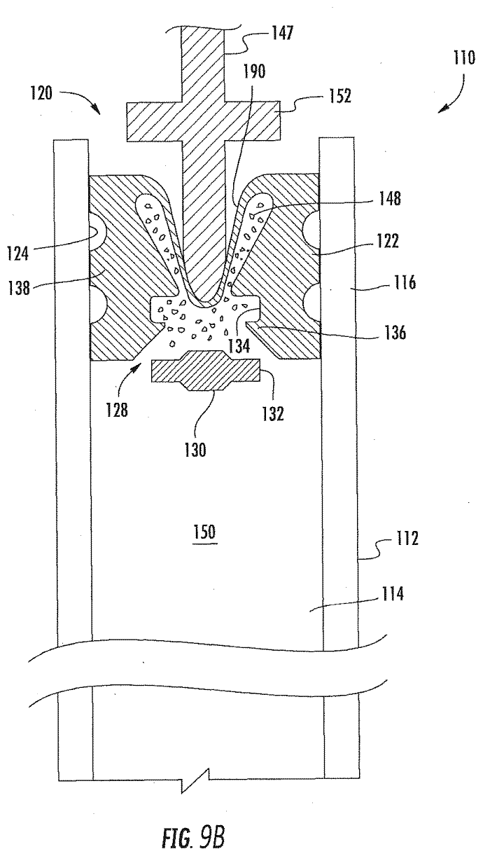

As shown in FIGS. 8, 9A, and 9B, mixing vial 110 is in the form of a cartridge and has a generally barrel shaped vessel 112 having a hollow interior chamber 114 and neck 116, and having distal and proximal open ends, 118 and 120. Neck 116 is a portion of vessel 112 at the proximal end thereof and is simply an extension of the vessel's tubular structure, the wall thickness, and diameter of vessel 112 not being reduced for neck 116. An elastomeric, chambered stopper-piston 122 is positioned in neck 116 and is in sealing relationship with interior facing wall 124 of neck 116.

Chambered stopper-piston 122 has a hollow interior chamber 126 and an open distal end 128 with a dislodgeable plug 130 positioned in and sealing said open end 128. Thus, annular edge 132 of plug 130 is tightly fit into annular groove 134 in radially inwardly extending stopper flange 136 of stopper 122. Vessel 112 may be made of glass, plastic or any other material suitable for use consistent with the purpose of the present invention. Stopper-piston 122 may be made of elastomeric plastic or rubber material which will seal well with adjacent surfaces. Plug 130 may be made of PTFE, or any material or combination of materials suitable for sealing with stopper-piston 122. Plug 130 may take the form of a sphere, convex disc, or other forms, and may be used for agitation of the chemicals to be mixed. Stopper piston 122 may have annular ribs 138 or other aids for sealing and stability. With respect to the embodiment shown in FIG. 9A, the plug 130 optionally may be made any material or combination of materials suitable for resisting penetration of the harpoon-plunger 146. For instance, the embodiment shown in FIG. 9A may include a plug 130 that may have an elastomeric body and edge, with a PTFE strike plate 131 attached to the harpoon side to prevent harpoon penetration.

As shown in FIGS. 8, 9A, and 9B, open distal end 118 of vessel 112 is sealed by rubber sealing cap 133 which is held in place by metal clip 140.

Referring to FIG. 9A, it is intended that, in use, cap 133 will be pierced by needle 142 when medical mixing vial, or cartridge, 110 is placed in syringe 57. Then, pushrod 146 is moved downwardly to first pierce stopper-piston 122 and then dislodge plug 130 as illustrated in FIG. 9A. Chemical reagent 148 such as buffering material in chamber 126 of stopper-piston 122 is thus allowed to mix with solution 150 in chamber 114. The mixture of drugs is then injected into a patient by manipulating pushrod 146 further downwardly, with shoulder 152 of pushrod 146 pushing against stopper-piston 122 to push stopper-piston 122 downwardly to act as a piston, sliding down vessel 112 and hydraulically expelling the liquid therein through needle 142.

Referring to FIG. 9B, it is intended that, in use, cap 133 will be pierced by needle 142 when medical mixing vial, or cartridge, 110 is placed in syringe 57. Then, blunt pushrod 147 is moved downwardly to first depress and elongate, but not penetrate, the membrane portion 190 of stopper-piston 122 and then dislodge plug 130, as illustrated in FIG. 9B. Chemical reagent 148 such as buffering material in chamber 126 of stopper-piston 122 is thus allowed to mix with solution 150 in chamber 114. The mixture of drugs is then injected into a patient by manipulating pushrod 147 further downwardly, with shoulder 152 of pushrod 147 pushing against stopper-piston 122 to push stopper-piston 122 downwardly to act as a piston, sliding down vessel 112 and hydraulically expelling the liquid therein through needle 142. Relaxation of the downward pressure on pushrod 147 causes a rebound of membrane 190, which causes aspiration of solution 150 back through the needle 142 and flow in the proximal direction.

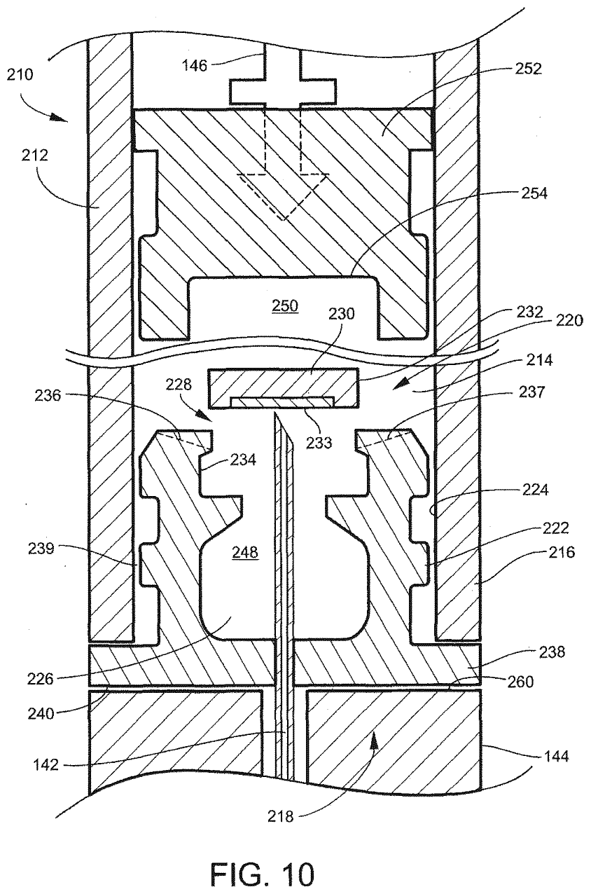

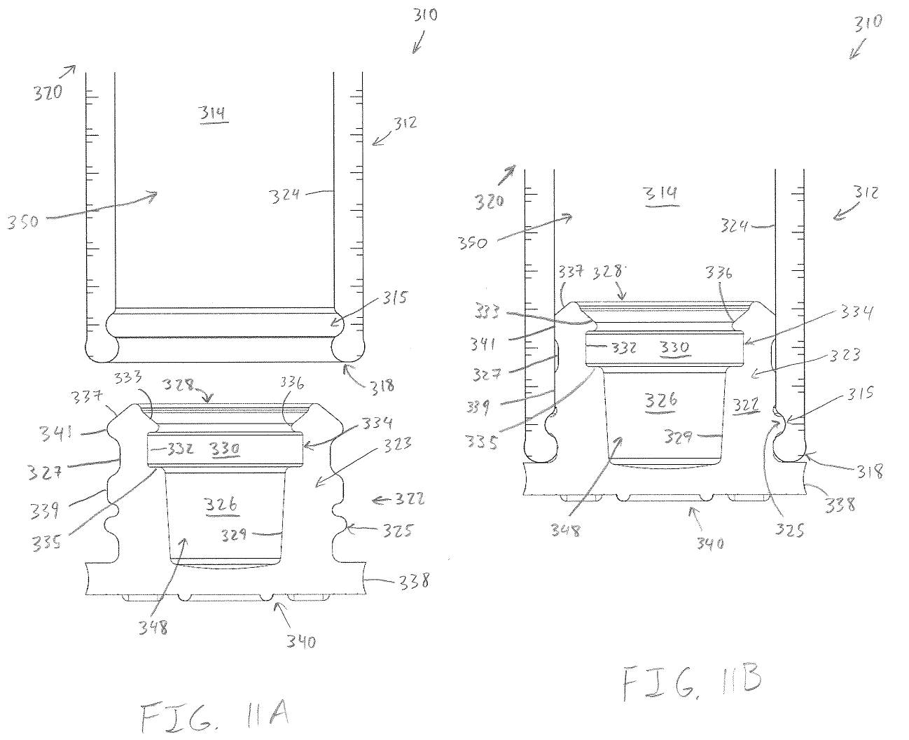

Another embodiment is shown in FIG. 10. As shown in FIG. 10, mixing vial 210 is in the general form of a syringe cartridge and has a generally barrel shaped vessel 212 having a hollow interior chamber 214 and neck 216 having distal and proximal open ends, 218 and 220. Neck 216 is a portion of vessel 212 at the distal end thereof and is simply an extension of the vessels tubular structure, as the wall thickness and diameter of vessel 212 are not changed for neck 216. An elastomeric, chambered stopper 222 is positioned in neck 216 and is in sealing relationship with interior facing wall 224 of neck 216. Stopper 222 has a radially outwardly extending annular flange 238 and ribs 239 and 241 and is retained in position in neck 216 during storage and transport by friction with vessel 212. During use, retention of stopper 222 in neck 216 is insured by proximally facing face 260 of syringe 57.

Chambered stopper 222 has a hollow interior chamber 226 and an open proximal end 228 with a dislodgeable plug 230 positioned in and sealing said open end 228. Thus, annular edge 232 of plug 230 is tightly fit into annular groove 234 in radially inwardly extending stopper flange 236 of stopper 222. The proximal face of stopper flange 236 may have radial grooves 237 to provide flow should plug 230 become lodged against proximal face of flange 236 after dislodgement from groove 234.

Vessel 212 may be made of glass, plastic or any other material suitable for use consistent with the purpose of the present invention. Stopper 222 may be made of an elastomeric plastic or rubber material, including the typical vial stopper material in present use, which will seal well with adjacent surfaces. Plug 230 may be made of PTFE, or any material or combination of materials suitable for sealing with stopper 222 and resisting penetration of the needle. For instance, plug 230 may have an elastomeric body and edge, with a PTFE strike plate 233 attached to the needle side to prevent needle penetration. Stopper 222 may have annular ribs or other sealing and retentive aids. Neck 216 may have inwardly facing grooves, ledges, or flanges to aid in sealing and retention of stopper 222.

Open proximal end 220 of vessel 212 is sealed by piston 252. Piston 252 is shown with a hollow recess 254 in the distal face which adds available volume inside the interior chamber 214 of the mixing vial 210 and prevents damage to the proximal end of needle 142 as piston 252 translates distally.

In use, it is intended that stopper 222 will be pierced by needle 142, as illustrated in FIG. 10 when mixing vial, or cartridge, 210 is placed in syringe 144. Then, needle 142 dislodges plug 230 from stopper 222, as proximally facing syringe face 260 abuts stopper distal face 240 and prevents hydraulic dislodgement. Chemical reagent 248 such as buffering material in chamber 226 of stopper 222 is thus allowed to mix with solution 250 such as local anesthetic in chamber 214. The mixture is then injected into a patient by manipulating syringe pushrod 146 distally against piston 252, hydraulically expelling the liquid through needle 142.

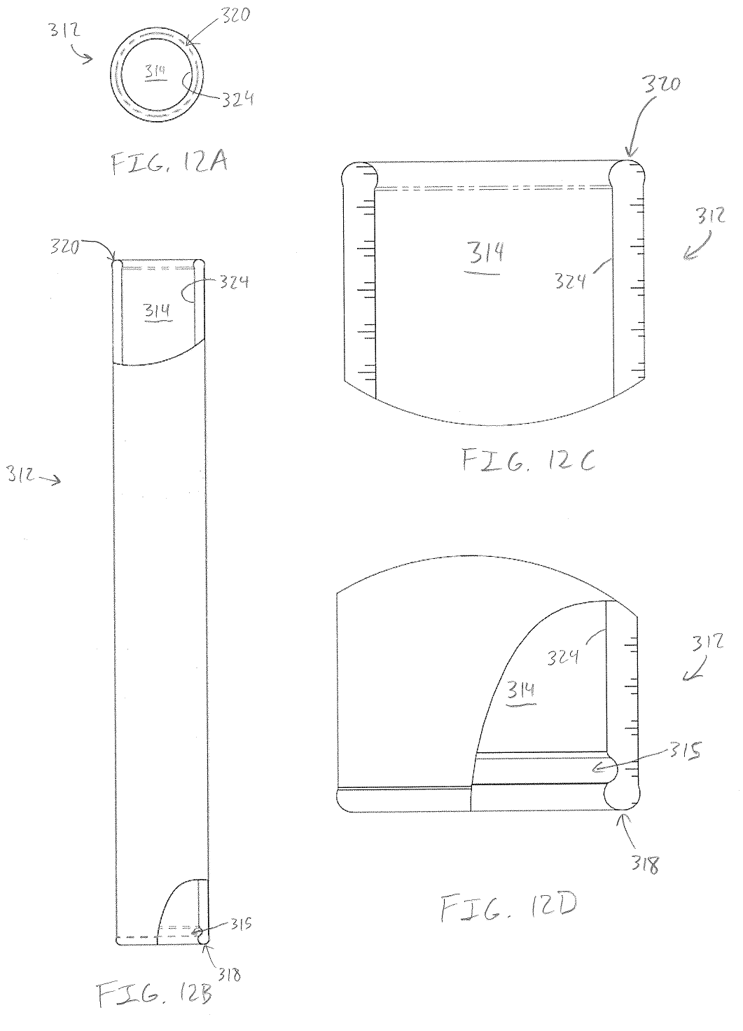

FIGS. 11A-13C show a mixing vial 310 having aspects according to various implementations. The mixing vial 310 shown in FIGS. 11A-13C is similar to those shown in FIGS. 1-10, and it is contemplated that features of the mixing vials 10, 110, 210 shown in FIGS. 1-10 can be incorporated in addition to, in combination with, and/or in replacement of one or more features of the mixing vial 310 shown in FIGS. 11A-13C to form various other implementations according to the disclosure herein.

The mixing vial 310 for use in a cartridge syringe 57 shown in FIGS. 11A-13C includes a vessel 312, a stopper 322, and a dislodgeable plug 330.

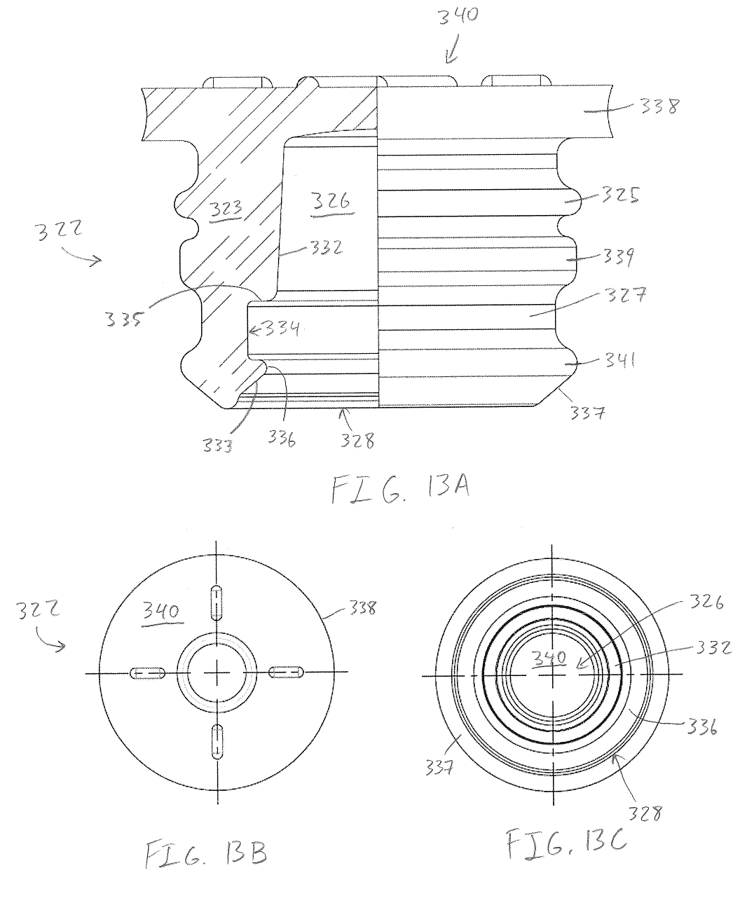

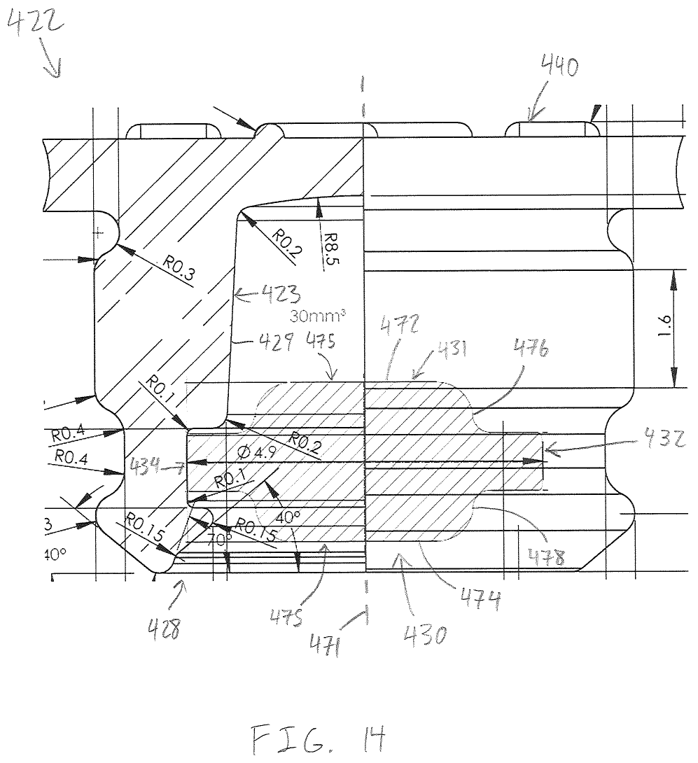

The vessel 312 has a proximal end 320 and a distal end 318 opposite and spaced apart from the proximal end 320. The vessel 312 also has an inner surface 324 extending from the proximal end 320 of the vessel 312 to the distal end 318 of the vessel 312 that defines a vessel interior chamber 314 extending from the distal end 318 toward the proximal end 320. The proximal end 320 and the distal end 318 of the vessel 312 have rounded edges to allow for easier insertion of a stopper 322 and/or a piston 252 during assembly. The vessel interior chamber 314 shown in FIGS. 11A-12D extends from the distal end 318 of the vessel 312 to the proximal end 320 of the vessel 312 to form a hollow tube, but in some implementations, the vessel interior chamber only extends partially from the distal end toward the proximal end such that the proximal end is a closed end.

The inner surface 324 of the vessel 312 defines an engagement groove 315 adjacent the distal end 318 of the vessel 312. For the vessel 312 shown in FIGS. 11A-12D, the distal end 318 of the vessel 312 is spaced apart from the engagement groove 315 by a distance of 1 mm. However, in some implementations, the distal end of the vessel is spaced apart from the engagement groove by a distance in the range of 4 mm or less, such as 3.75 mm or less, 3.5 mm or less, 3.25 mm or less, 3.0 mm or less, 2.75 mm or less, 2.5 mm or less, 2.25 mm or less, 2.0 mm or less, 1.75 mm or less, 1.5 mm or less, 1.25 mm or less, 1.0 mm or less, 0.75 mm or less, 0.5 mm or less, or 0.25 mm or less.

The engagement groove 315 shown in FIGS. 11A-12D fully extends circumferentially around the inner surface 324 of the vessel 312 to form an annular engagement groove 315. However, in some implementations, the engagement groove extends circumferentially at least partially around the inner surface of the vessel.

The vessel 312 has an interior diameter as measured from the inner surface 324 of the vessel 312 across the vessel interior chamber 314. The interior diameter is uniform from the engagement groove 315 to the proximal end 320 of the vessel 312 and from the engagement groove 315 to the distal end 318 of the vessel 312. Although the interior diameter from the engagement groove 315 to the proximal end 320 of the vessel 312 and the interior diameter from the engagement groove 315 to the distal end 318 of the vessel 312 in FIGS. 11A-12D are equal, in some implementations, the interior diameter from the engagement groove to the proximal end of the vessel is a uniform first diameter and the interior diameter from the engagement groove to the distal end of the vessel is a uniform second diameter that is different from the first diameter. In some implementations, the interior diameter from the engagement groove to the proximal end of the vessel is nonuniform and/or the interior diameter from the engagement groove to the distal end of the vessel is nonuniform.

The stopper 322 includes a side wall 323 having an open first end 328 and a closed second end 340 opposite and spaced apart from the open first end 328. The stopper 322 further has an outer surface 327 extending between the open first end 328 and the closed second end 340 and an inner surface 329 radially spaced apart from the outer surface 327 of the stopper 322. The inner surface 329 and the closed second end 340 of the stopper 322 define a stopper interior chamber 326. The outer surface 327 of the stopper 322 at the open first end 328 defines a taper 337 to allow for easier insertion of the stopper 322 into the vessel interior chamber 314 during assembly.

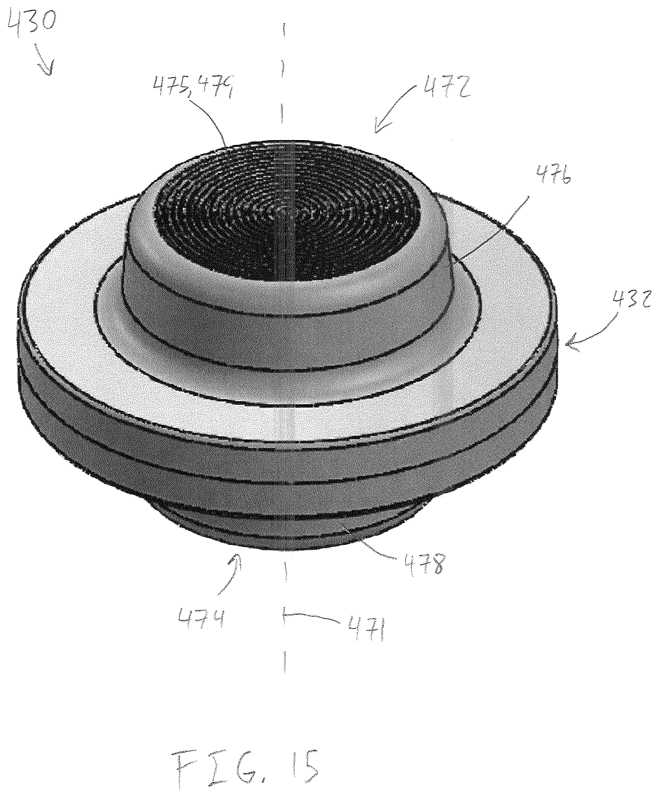

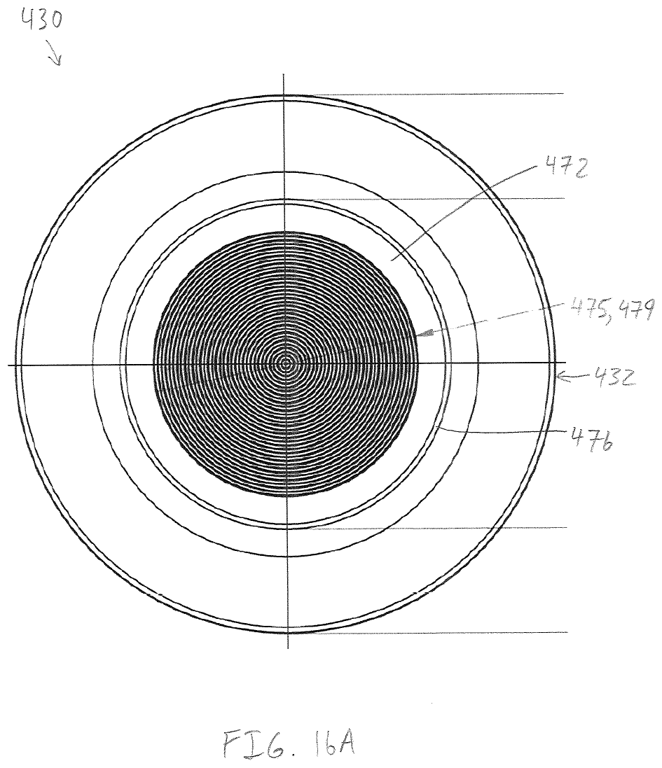

The stopper 322 shown in FIGS. 11A, 11B, and 13A-13C includes an elastomeric plastic or rubber, but in some implementations, the stopper includes any resilient material capable of being penetrated by a needle. The closed second end 340 of the stopper 322 and the side wall 323 shown in FIGS. 11 and 13A-13C are formed as a single piece. However, in some implementations, the closed second end of the stopper and the side wall are formed separately and coupled to each other by known means.

The inner surface 329 of the stopper 322 includes a radially inwardly extending flange 336 adjacent the open first end 328. The radially inwardly extending flange 336 has a first flange portion 333 and a second flange portion 335 axially spaced apart from the first flange portion 333. The first flange portion 333 is axially closer than the second flange portion 335 to the open first end 328 of the stopper 322. The first flange portion 333 and the second flange portion 335 of the radially inwardly extending flange 336 of the inner surface 329 of the stopper 322 defines an annular stopper groove 334 for receiving the edge 332 of the dislodgeable plug 330, as discussed below. The radially inwardmost edge of the first flange portion 333 has a first diameter, and the radially inwardmost edge of the second flange portion 335 has a second diameter. The first diameter is larger than the second diameter.

The different diameter flange portions 333, 335 that define the inward facing annular stopper groove 334 allow for easier insertion of a dislodgeable plug 330 into the annular stopper groove 334 without over inserting the dislodgeable plug 330 into the stopper interior chamber 326. When the annular stopper groove is simply defined by the side wall of the stopper, the plug must exert compressive forces on the end of the side wall of the stopper to deform the side wall enough to allow the plug to be inserted into the groove.

For an annular stopper groove defined by a flange that extends radially inwardly from the side wall of the stopper, the plug exerts shear and tensile forces on the cantilevered flanges, which requires less force from the user during insertion. While this makes it easier for insertion into the annular stopper groove, it can also make it easier to over-insert the plug past the groove and into the stopper interior chamber. Inclusion of a second flange portion that has a smaller diameter minimizes the chances of the user overexerting the plug past the annular stopper groove and into the stopper interior chamber.

Although the stopper 322 shown in FIGS. FIGS. 11A, 11B, and 13A-13C includes a radially inwardly extending flange 336, in some implementations, the stopper does not include a radially inwardly extending flange and the inner surface of the stopper defines an annular stopper groove.

The outer surface 327 of the stopper 322 includes two ribs 339, 341 extending radially outwardly from the outer surface 327 of the stopper 322. Each of the two ribs 339, 341 are axially spaced apart from each other. The two ribs 339, 341 are configured to form a sealing relationship with a portion of the inner surface 324 of the vessel 312 when the stopper 322 is disposed within the vessel interior chamber 314 to prevent leaking of the contents of the mixing vial 310 and to prevent axial movement of the stopper 322 when disposed within the vessel 312. Although the outer surface 327 of the stopper 322 shown in FIGS. 11 and 13A-13C includes two ribs 339, 341, in some implementations, the stopper includes any number of one or more ribs, such as two or more ribs, three or more ribs, four or more ribs, five or more ribs, six or more ribs, seven or more ribs, eight or more ribs, nine or more ribs, or ten or more ribs.

The stopper 322 further includes an engagement protrusion 325 extending radially away from the outer surface 327 of the stopper 322. The engagement protrusion 325 is disposable within the engagement groove 315 when the stopper 322 is disposed within the vessel interior chamber 314 and is in a sealing relationship with a portion of the inner surface 324 of the vessel 312. The closed second end 340 of the stopper 322 further includes a radially outwardly extending annular flange 338 that is configured to abuts the distal end 318 of the vessel 312 when the engagement protrusion 325 is disposed within the engagement groove 315. The engagement of the engagement protrusion 325 with the engagement groove 315 secures the stopper 322 within the distal end 318 of the vessel interior chamber 314 during operation.

The engagement protrusion 325 shown in FIGS. 11 and 13A-13C is spaced apart from the radially outwardly extending annular flange 338 by a distance of 1.0 mm to match the distance from the distal end 318 of the vessel 312 to the engagement groove 315. However, in some implementations, the engagement protrusion is spaced apart from the radially outwardly extending annular flange by a distance in the range of 4 mm or less, such as 3.75 mm or less, 3.5 mm or less, 3.25 mm or less, 3.0 mm or less, 2.75 mm or less, 2.5 mm or less, 2.25 mm or less, 2.0 mm or less, 1.75 mm or less, 1.5 mm or less, 1.25 mm or less, 1.0 mm or less, 0.75 mm or less, 0.5 mm or less, or 0.25 mm or less.

The engagement protrusion 325 shown in FIGS. 11A, 11B, and 13A-13C fully extends circumferentially around the outer surface 327 of the stopper 322 to form an annular engagement protrusion 325. However, in some implementations, the engagement protrusion extends circumferentially at least partially around the outer surface of the stopper. In some implementations, the engagement groove fully extends circumferentially around the inner surface of the vessel and the engagement protrusion extends circumferentially at least partially around the outer surface of the stopper. In such implementations, the engagement protrusion can still be disposed within the annular engagement groove, but the user does not have to “clock,” or angularly orient, the engagement protrusion with the engagement groove since the engagement groove extends circumferentially around the inner surface of the vessel.

The inner surface 324 of the vessel 312 shown in FIGS. 11-13C defines the engagement groove 315 and the engagement protrusion 325 extends radially away from the outer surface 327 of the stopper 322, however, in some implementations, the outer surface of the stopper defines the engagement groove and the engagement protrusion extends radially away from inner surface of the vessel such that the engagement protrusion is disposable within the engagement groove when the stopper is disposed within the vessel interior chamber and is in a sealing relationship with a portion of the inner surface of the vessel. In such implementations, the dimensions, locations, and uses of the engagement groove and the engagement protrusion can be the same as those described above with respect to the implementation shown in FIGS. 11-13C.

FIGS. 11A and 11B shows the dislodgeable plug 330 disposed within the stopper interior chamber 326 such that at least a portion of an edge 332 of the dislodgeable plug 330 is disposed within the annular stopper groove 334. The entire dislodgeable plug 330 is fully dislodgeable as a single piece from the open first end 328 of the stopper 322.

In FIGS. 11A and 11B, an anesthetic 350 is disposed within the vessel interior chamber 314, and a buffering material 348 is disposed within the stopper interior chamber 326. The dislodgeable plug 330 separates the anesthetic 350 from the buffering material 348. As in the implementation shown in FIG. 10, the mixing vial 312 shown in FIGS. 11A-12D can further include a piston 252 slidably positioned within the vessel interior chamber 314. Similar to the piston 252 shown in FIG. 10, the piston 252 can define a hollow recess 254 in a distal face to allow for the dislodgeable plug 330 to enter the recess 254 once dislodged from the stopper 322.

The mixing vial 310 can be used with a cartridge syringe 57 such as the dental syringe shown in FIGS. 5 and 6. As described in detail above, such a syringe 57 includes a body 58 having a distal end 60, a syringe needle 142, and an axially movable plunger 146. The syringe needle 142 has a distal end 143 and a proximal end 144, and the syringe needle 142 is statically coupled to the distal end 60 of the body 58 of the syringe 57. When the mixing vial 310 is loaded into or disposed in the syringe 57, the proximal end of the syringe needle 142 is able to penetrate the closed second end 340 of the stopper 322. The proximal end 144 of the needle 142 passes through the stopper interior chamber 326 and contacts the dislodgeable plug 330, which fully dislodges the dislodgeable plug 330 from the stopper 322. The anesthetic 350 disposed within the vessel interior chamber 314 and the buffering material 348 disposed within the stopper interior chamber 326 are then able to mix.

Various implementations include a mixing vial. The vial includes a vessel, a stopper, and a dislodgeable plug. The vessel includes a hollow interior chamber having a proximal end, a distal end, and a neck adjacent the proximal end. The neck has a distal end and a proximal open end. The stopper includes a hollow interior chamber with an open distal end and a closed proximal end. The dislodgeable plug has a body and a radially outwardly extending flange. The body has a longitudinal axis, a first axial end, and a second axial end opposite and spaced apart along the longitudinal axis from the first end. The dislodgeable plug is disposed within and sealing the open distal end of the stopper. The entire dislodgeable plug is fully dislodgeable as a single piece from the open distal end of the stopper-piston. The first axial end includes a textured surface.

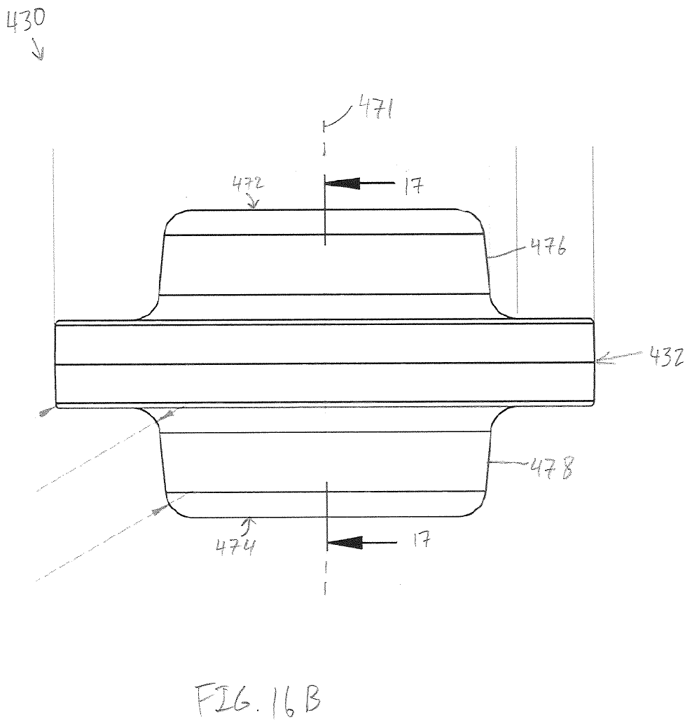

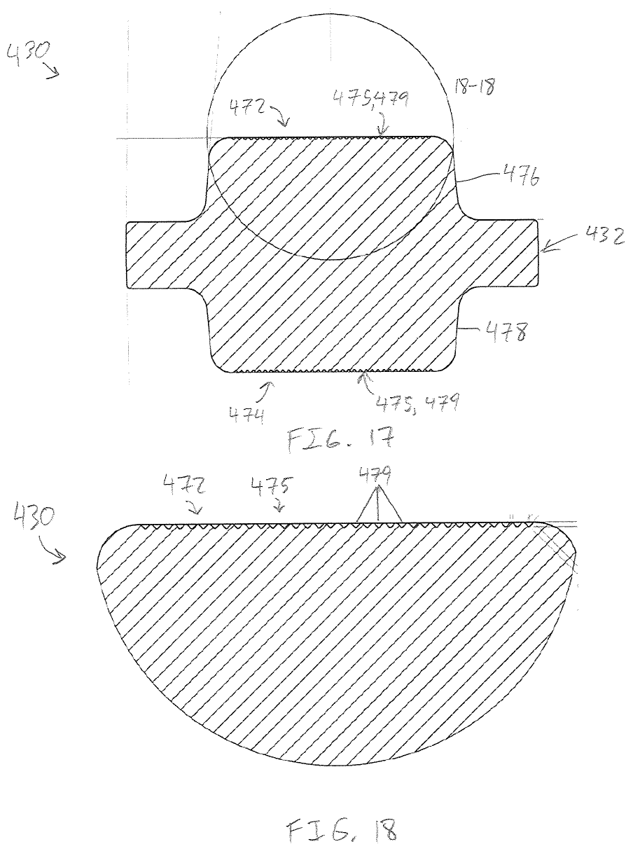

FIGS. 14-18 show a dislodgeable plug 430, according to another implementation, that is disposable within any of the stoppers or stopper-pistons disclosed herein and used in conjunction with any of the mixing vial systems disclosed here.

The dislodgeable plug 430 includes a body 431 and a radially outwardly extending flange 432. The body 431 has a longitudinal axis 471, a first axial end 472, and a second axial end 474 opposite and spaced apart along the longitudinal axis 471 from the first end 472.

The radially outwardly extending flange 432 of the dislodgeable plug 430 is disposed between and is equally spaced from the first axial end 472 and the second axial end 474 of the body 431. However, in some implementations, the radially outwardly extending flange can be disposed closer to one of the first axial end or the second axial end of the body. In some implementations, the radially outwardly extending flange can be disposed at one of the first axial end or the second axial end of the body.

As with other implementations disclosed herein, the dislodgeable plug 430 is able to be disposed within and seal the open distal end 428 of the stopper 422. The entire dislodgeable plug 430 is fully dislodgeable as a single piece from the open distal end 428 of the stopper 422.

A first portion 478 of the body 431 of the dislodgeable plug 430 between the first axial end 472 and the radially outwardly extending flange 432 is tapered. The plug 430 further includes a concave filleted transition between the radially outwardly extending flange 432 and the first portion 476 and a convex filleted transition between the first portion 476 and the first axial end 472.

The largest cross-sectional area of the first portion 476 is located adjacent the radially outwardly extending flange 432 and the smallest cross-sectional area of the first portion 476 is located adjacent the first axial end 472. The first portion 476 of the dislodgeable plug 430 shown in FIGS. 14-18 has a circular cross-section when viewed in a plane perpendicular to the longitudinal axis 471. Thus, the largest cross-sectional area of the first portion 476 is also a largest diameter of the first portion 476, and the smallest cross-sectional area of the first portion 476 is the smallest diameter of the first portion 476. The entire axial length of the first portion 476 of the dislodgeable plug 430 shown in FIGS. 14-18 includes a continuous and consistent taper. However, in some implementations, the first portion can include two or more tapered sections with no taper in between. In some implementations, the first portion can have a varying taper along its axial length. In some implementations, the dislodgeable plug does not have a concave filleted transition between the radially outwardly extending flange and the first portion, a convex filleted transition between the first portion and the first axial end, or either filleted transition such that the first portion extends to the radially outwardly extending flange, the first axial end, or both, respectively. In some implementations, the transition between the first portion and the first axial end is a chamfered transition. In some implementations, the transition between the first portion and the first axial end is a chamfered transition.

The dislodgeable plug 430 also includes a tapered second portion 478 of the body 431 of the dislodgeable plug 430 between the second axial end 474 and the radially outwardly extending flange 432. The plug 430 further includes another concave filleted transition between the radially outwardly extending flange 432 and the second portion 478 and another convex filleted transition between the second portion 478 and the second axial end 474.

The largest cross-sectional area of the second portion 478 is located adjacent the radially outwardly extending flange 432 and the smallest cross-sectional area of the second portion 478 is located adjacent the second axial end 474. The second portion 478 of the dislodgeable plug 430 shown in FIGS. 14-18 has a circular cross-section when viewed in a plane perpendicular to the longitudinal axis 471. Thus, the largest cross-sectional area of the second portion 478 is also a largest diameter of the second portion 478, and the smallest cross-sectional area of the second portion 478 is the smallest diameter of the second portion 478. The entire axial length of the second portion 478 of the dislodgeable plug 430 shown in FIGS. 14-18 includes a continuous and consistent taper. However, in some implementations, the second portion can include two or more tapered sections with no taper in between. In some implementations, the second portion can have a varying taper along its axial length. In some implementations, the dislodgeable plug does not have a concave filleted transition between the radially outwardly extending flange and the second portion, a convex filleted transition between the second portion and the second axial end, or either filleted transition such that the second portion extends to the radially outwardly extending flange, the second axial end, or both, respectively. In some implementations, the transition between the second portion and the second axial end is a chamfered transition. In some implementations, the transition between the second portion and the second axial end is a chamfered transition.

The tapered first portion 476 and the tapered second portion 478 of the body 431 aid the dislodgeable plug 430 in being inserted into, and dislodged from, the inwardly facing annular groove 434 of the stopper 422. During insertion and dislodgement, a side wall of the plug 430 can rub against the inner side walls 429 of the stopper 422. The friction from the contact of these walls can require additional force to insert the plug 430 into, and dislodge the plug 430 from, the stopper 422. This additional required force can create an imbalance between the insertion/dislodgement force and the amount of force necessary to cause the stopper 422 to move.

In implementations in which the stopper is a stopper-piston, it is desired that the force from the harpoon of a syringe dislodge the plug before causing the stopper-piton to move axially toward the distal end of the vessel. Reducing the frictional forces of dislodging the plug from the stopper can cause the plug to be dislodged with less force than translation of the stopper, resulting in the plug being dislodged from the harpoon first and the stopper moving toward the distal end thereafter.

In implementations in which the stopper is disposed at the distal end of the vessel, reducing the frictional forces of inserting and dislodging the plug from the stopper can reduce the chances of the stopper moving during use, which can result in leaking of the mixing vial.

The frictional forces between the stopper walls 429 and the dislodgeable plug 430 are further increased when the stopper 422 is disposed within a vessel when the dislodgeable plug 430 is being inserted. When a resilient stopper 422 is disposed within the vessel, expansion of the walls 423 of the stopper 422 is limited by the walls of the vessel that surround the stopper 422. The radially extending flange 432 of the dislodgeable plug 430 has a larger diameter than the inner diameter of the side walls 423 of the stopper 422, which allows the stopper 422 to retain the radially extending flange 432 within the inwardly facing annular groove 434 of the stopper 422. Thus, as the dislodgeable plug 430 is being inserted into the stopper 422, the side walls 423 of the stopper 422 typically deform to allow the radially extending flange 432 to pass through the stopper 422 and into the inwardly facing annular groove 434. When the side walls 423 of the stopper 422 are restricted from expanding, additional force is required to insert the plug 430 into, and dislodge the plug 430 from, the stopper 422. Reducing the friction between the stopper walls 423 and the dislodgeable plug 430 by including a tapered first portion 476 and/or second portion 478 minimizes the amount of force necessary to insert and dislodge the plug 430.

The largest cross-sectional area of the first portion 476 and/or second portion 478 of the of the dislodgeable plug 430 is sized such that at least a portion of the first portion 476 and/or the second portion 478 of the body 431 of the dislodgeable plug 430 can create a friction fit between the stopper side walls 423 and the plug 430. This can ensure that the dislodgeable plug 430 creates a water-tight seal between the stopper 422 and the plug 430 during use.

The smallest cross-sectional area of the first portion 476 and/or second portion 478 of the dislodgeable plug 430 is sized such that at least a portion of the first portion 476 and/or the second portion 478 of the body 431 of the dislodgeable plug 430 does not contact the stopper side walls 423 when the plug 430 is inserted into the stopper 422. As discussed above, this reduces the frictional forces between the plug 430 and the stopper 422. However, the smallest cross-sectional area is configured to be large enough such that the harpoon 146 or syringe needle 55 has enough surface area at the first axial end 472 and/or the second axial end 474 to press against without deflecting or slipping off of the first axial end 472 and/or the second axial end 474 when force is applied during dislodgement.

The first axial end 472 and the second axial end 474 of the dislodgeable plug 430 shown in FIGS. 14-18 include a textured surface 475. However, in some implementations, only one of the axial ends includes a textured surface. In some implementations, neither of the axial ends includes a textured surface.

The textured surface 475 shown in FIGS. 14-18 includes a plurality of grooves 479. The grooves 479 are configured as concentric circle grooves 479. When a harpoon 146 or needle 55 from a syringe penetrates the closed proximal end 440 of the stopper 422 and contacts the first axial end 472 or the second axial end 474 of the dislodgeable plug 430, the grooves 479 of the textured surface 475 help prevent the harpoon 146 or needle 55 of a syringe from slipping or deflecting. This allows the force from the harpoon 146 or needle 55 to be directed to the plug 430. The concentric circle pattern of the grooves 479 allow the harpoon 146 or needle 55 to enter a groove 479 but prevent the harpoon 146 or needle 55 from moving radially outwardly. However, in some implementations, the textured surface includes any number of concentric grooves or grooves that have the same center point. In some implementations, the textured surface can include a single spiral groove that includes two or more groove portions that are radially spaced apart from each other. In some implementations, the textured surface includes two or more groove portions that are arcuate and radially spaced apart from each other. In some implementations, the textured surface includes two or more groove portions that extend parallel to a perimetrical edge of the first axial end or the second axial end. In some implementations, the textured surface includes two or more groove portions that are arcuate.

In some implementations, the textured surface can define a concave portion. The concave portion acts as a single circular groove to prevent a harpoon or needle from deflecting or slipping from the first axial end or the second axial end. In some implementations, the textured surface can define two or more concave portions. In some implementations, the textured surface can include a rough surface or a resilient layer to prevent a harpoon or needle from deflecting or slipping from the first axial end or the second axial end.

In some implementations, one or both of the first axial end and/or the second axial end include a strike plate, such as those described herein with regards to the implementations shown in FIGS. 3, 4, 8, 9A, and 10. In such implementations, the dislodgeable plug is disposed within the open distal end of the stopper-piston such that the strike plate faces the hollow interior chamber of the stopper-piston. The strike plate is able to resist penetration by the harpoon when the harpoon is used to dislodge the dislodgeable plug such that the harpoon can penetrate the closed proximal end of the stopper but does not penetrate the dislodgeable plug.

In some implementations, the dislodgeable plug can be used in a system in conjunction with any other implementation of a syringe, needle, or other apparatus for dislodging a dislodgeable plug from a stopper described herein or known in the art. In some implementations, the dislodgeable plug can be used in conjunction with any other implementation of a stopper or stopper-piston described herein and in conjunction with any other implementation of a vessel described herein. In implementations in which the dislodgeable plug is used in conjunction with a stopper-piston implementation, the entire stopper-piston may be slidably positioned in a sealing relationship with the neck. In such implementations, the entire stopper-piston is slidable from a first position to a second position in which the entire stopper-piston is adjacent the proximal end of the vessel when in the first position and the entire stopper-piston is closer to the distal end of the vessel than to the proximal end of the vessel when in the second position.

Various other implementations include a dislodgeable plug 530 that seals the open distal end of a stopper, such as any of those described herein. This plug 530 has a body 575 with a longitudinal axis 571 and opposing first and second axial ends 572, 574. The plug body 575 also includes a radially outwardly extending flange 532. The entire dislodgeable plug 530 is designed to be fully removable from the stopper as a single intact piece.

The dislodgeable plug 530 isolates the interior chambers of the vessel and stopper which may contain different substances that are kept separate until mixing is desired. For example, the vessel chamber may hold an anesthetic solution while the stopper chamber contains a buffering reagent. When the plug 530 is dislodged, these components can mix together prior to injection.

This syringe system allows for convenient storage of multiple components and on-demand mixing immediately before use. The dislodgeable plug 530 provides an effective seal between the chambers while also being easily removable when needed. The elastomeric stopper helps maintain proper sealing within the vessel neck.

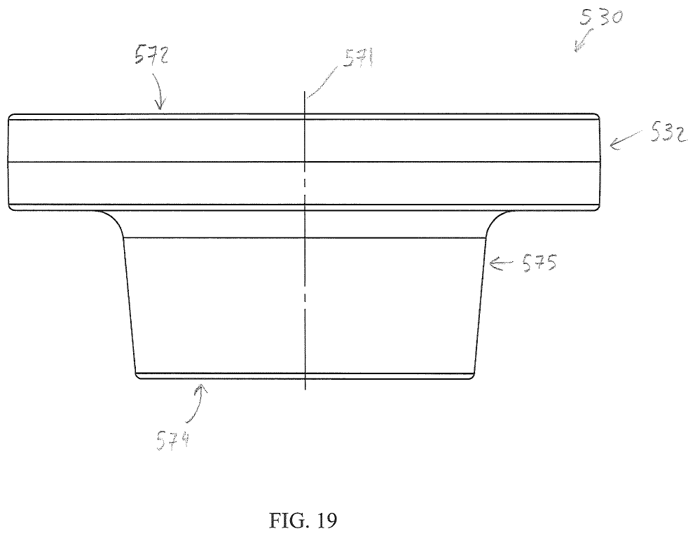

FIGS. 19-21 illustrate another implementation of a dislodgeable plug component 530 that may be used in combination with any of the stoppers or stopper-pistons disclosed herein.

The dislodgeable plug 530 shown in FIGS. 19-21 includes a body 575 and a radially outwardly extending flange 532. The body 575 has a longitudinal axis 571, a first axial end 572, and a second axial end 574 opposite and spaced apart along the longitudinal axis 571 from the first axial end 572.

In this implementation, the radially outwardly extending flange 532 is disposed at the first axial end 572 of the dislodgeable plug 530. This configuration differs from some previously described implementations where the flange 532 may be positioned between the first and second axial ends 572, 574.

A portion of the body 575 of the dislodgeable plug 530 between the second axial end 574 and the radially outwardly extending flange 532 is tapered. This tapered portion has a largest cross-sectional area adjacent the radially outwardly extending flange 532 and a smallest cross-sectional area adjacent the second axial end 574. The tapered design may facilitate insertion and removal of the plug 530 from a stopper.

FIG. 19 shows a section view of the dislodgeable plug 530. The plug 530 has a frustoconical body 575 with the tapered profile visible. At the first axial end 572, there is a wider section forming the radially outwardly extending flange 532. The body 575 tapers from this flange 532 toward the second axial end 574, creating a gradually decreasing cross-sectional area.

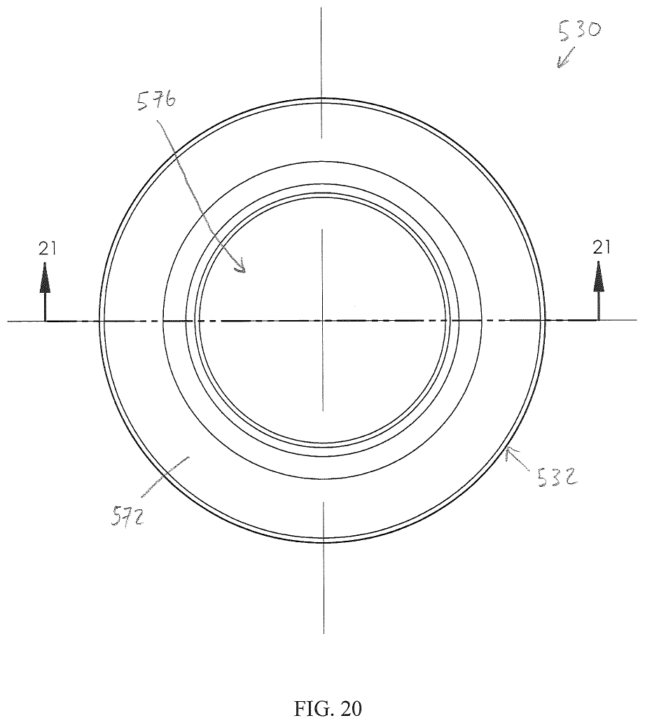

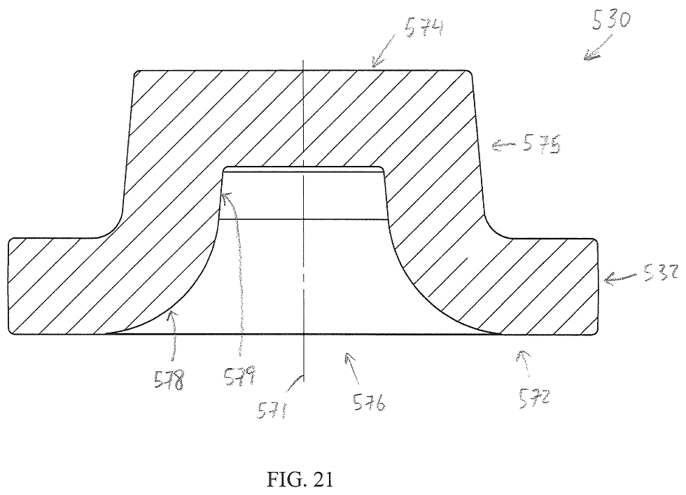

FIG. 20 illustrates a top view of the first axial end 572 of the dislodgeable plug 530. In this view, a reference line 21-21 is shown, indicating the plane along which the section view of FIG. 21 is taken.

FIG. 21 depicts a section view of the dislodgeable plug 530 along the reference line 21-21 shown in FIG. 20. This cross-sectional view reveals the internal geometry and proportions of the plug component. The cross-hatching indicates the solid portions of the plug structure.

The dislodgeable plug 530 shown in FIGS. 19-21 may be asymmetrical across a plane perpendicular to the longitudinal axis 571 of the plug 530 and centered on the flange 532. This asymmetry contrasts with some previously described symmetrical plug designs.

In some cases, the second axial end 574 of the dislodgeable plug 530 may include a strike plate implemented similarly to the strike plates described with respect to other plugs disclosed herein. When the plug 530 is disposed within the open distal end of a stopper, the strike plate may face the hollow interior chamber of the stopper. The strike plate may be configured to resist penetration by a harpoon or needle when used to dislodge the plug 530.

The dislodgeable plug 530 may be designed such that, when positioned on its side on a level surface with the radially outwardly extending flange 532 and the edge of the second axial end 574 touching the surface, the center of mass causes the plug 530 to tip over onto its second axial end 574. The plug 530 can also rest on its first axial end 572. Thus, the plug 530 has only two static resting positions--on its first axial end 572 and on its second axial end 574. As used herein, the term “static resting position” is used to describe a position in which the plug can remain static without outside influences on a flat surface that is perpendicular to a gravitational axis. This feature may be beneficial in manufacturing and assembly processes by ensuring the plug 530 rests in a predictable orientation.

The first axial end 572 of the plug body 575 may define a plug opening 576 extending from the first axial end 572 toward the second axial end 574. The side walls of this plug opening 576 may be tapered, with a larger diameter adjacent the first axial end 572 than adjacent the second axial end 574. This tapered opening 576 may serve multiple purposes, including shifting the center of mass toward the second axial end 574 and providing a suitable interface for automated manufacturing tools.

The dislodgeable plug 530 shown in FIGS. 19-21 may be used in conjunction with any of the mixing vial systems described throughout the application. For example, it may be inserted into the open end of a stopper or stopper-piston, which in turn may be positioned within the neck of a vessel containing an anesthetic solution or other substance. The plug 530 may separate the contents of the vessel's interior chamber from a buffering reagent or other material contained within the stopper chamber.

When used in a mixing vial system, the dislodgeable plug 530 may be fully removed as a single piece from the open end of the stopper when activated by a needle or harpoon mechanism. This allows the separated substances to mix prior to administration.

The first axial end 572 of the dislodgeable plug 530 defines a plug opening 576 extending from the first axial end 572 toward the second axial end 574, as shown in FIG. 20 and FIG. 21. The side walls of the plug opening 576 are tapered such that the plug opening 576 has a larger diameter adjacent the first axial end 572 than it does adjacent the second axial end 574. This tapered configuration serves multiple purposes.

In some cases, the taper of the side walls of the plug opening 576 includes a constant taper portion 579 and a varying taper portion 578. The varying taper portion 578 may be disposed adjacent the first axial end 572 and the constant taper portion 579 may be disposed adjacent the second axial end 574. FIG. 21 illustrates this configuration in cross-section, showing how the taper profile changes along the length of the plug opening 576.

The varying taper portion 578 adjacent the first axial end 572 may have a curved or non-linear profile. This varying taper 578 may provide a smooth transition from the larger diameter at the first axial end 572 to the constant taper portion 579. In some cases, the varying taper portion 578 may help guide insertion tools or manufacturing equipment during assembly processes.

The constant taper portion 579 adjacent the second axial end 574 may have a linear profile with a consistent angle relative to the longitudinal axis 571. This constant taper 579 may provide predictable dimensional changes along its length, which may be beneficial for certain manufacturing or functional requirements.

The larger diameter of the plug opening 576 adjacent the first axial end 572 may facilitate easier insertion of tools or components during manufacturing or assembly. In some cases, the larger opening 576 may also help shift the center of mass of the dislodgeable plug 530 toward the second axial end 574, which may contribute to the plug's stability characteristics.

The smaller diameter of the plug opening 576 adjacent the second axial end 574 may help maintain structural integrity of the dislodgeable plug 530 while still allowing for sufficient material removal to achieve desired weight distribution or other functional properties.

In some cases, the tapered side walls of the plug opening 576 may be designed to accommodate specific tools or components used in the manufacturing or assembly process of the mixing vial. The taper may help center or align these tools as they are inserted into the plug opening 576.

The combination of varying and constant taper portions 578, 579 in the plug opening 576 may provide a balance of functional benefits. The varying taper 578 may offer improved insertion characteristics and stress distribution, while the constant taper 579 may provide more predictable dimensional relationships for precise fit or interaction with other components of the mixing vial system.

In some implementations, the plug opening 576 may include only a constant taper portion 579 or only a varying taper portion 578 along its side walls. Alternatively, the plug opening 576 may have no taper at all, maintaining a uniform diameter from the first axial end 572 toward the second axial end 574. The choice of taper configuration may depend on specific design requirements, manufacturing processes, or intended interactions with other components of the mixing vial system. For instance, a constant taper 579 may be preferred for precise dimensional control, while a varying taper 578 might offer advantages in stress distribution or tool insertion. A non-tapered opening could be suitable in cases where a straight bore is desired for particular functional or assembly needs. In some implementations, the taper of the side walls of the plug opening 576 may include a constant taper portion 579 disposed adjacent the first axial end 572 and a varying taper portion 578 disposed adjacent the second axial end 574.

The dislodgeable plug 530 shown in FIGS. 19-21 includes an asymmetrical design that contributes to specific static resting positions. The body 575 of the dislodgeable plug 530 has a longitudinal axis 571, a first axial end 572, and a second axial end 574 opposite and spaced apart along the longitudinal axis 571 from the first axial end 572. A radial flange 532 extends outwardly from the body 575 at the first axial end 572.

The asymmetrical design of the dislodgeable plug 530 results in a center of mass that may be positioned closer to the second axial end 574 than to the first axial end 572. This asymmetry and center of mass location contribute to the dislodgeable plug 530 having two distinct static resting positions.

In some cases, when the dislodgeable plug 530 is placed on a level surface such that the radial flange 532 and an edge of the second axial end 574 are in contact with the surface, the center of mass causes the dislodgeable plug 530 to tip over onto the second axial end 574. This creates a first static resting position where the dislodgeable plug 530 rests on the second axial end 574.

The second static resting position occurs when the dislodgeable plug 530 is placed on the first axial end 572, with the radial flange 532 in contact with the surface. In this position, the dislodgeable plug 530 remains stable due to the larger surface area provided by the radial flange 532.

The two static resting positions of the dislodgeable plug 530 may provide benefits for manufacturing and assembly processes. By ensuring that the dislodgeable plug 530 consistently rests in one of two predictable orientations, automated machinery may more easily handle and orient the component during production and assembly of the mixing vial. This feature may reduce the complexity of manufacturing equipment and potentially increase production efficiency.

In some cases, the asymmetrical design and resulting static resting positions may also facilitate visual inspection during quality control processes, as the orientation of the dislodgeable plug 530 may be quickly determined. Also, because the surfaces on which the plug 530 rests in both resting positions are flat, the plug 530 is not prone to rolling in either of its resting positions.

The combination of the asymmetrical design, center of mass location, and resulting static resting positions contributes to a dislodgeable plug 530 that may be more easily manufactured, assembled, and integrated into the mixing vial system.

The dislodgeable plug 530 shown in FIGS. 19-21 includes several interacting elements that facilitate proper positioning, assembly, and operation within the mixing vial systems described throughout the application.

The radial flange 532 is configured to engage with an inwardly facing annular groove of the stopper. The stopper may define a radially inwardly extending flange, wherein the radially inwardly extending flange defines the inwardly facing annular groove. This engagement helps secure the dislodgeable plug 530 within the stopper and provides a seal between the two components.

The body 575 of the dislodgeable plug 530 includes a portion between the second axial end 574 and the radial flange 532 that may be tapered. This tapered portion has a largest cross-sectional area adjacent the radial flange 532 and a smallest cross-sectional area adjacent the second axial end 574. The tapered design may facilitate insertion of the dislodgeable plug 530 into the stopper and may also aid in the dislodging process when activated.

In some implementations, the plug 530 shown in FIGS. 19-21 can include any other of the features disclosed with respect to any other dislodgeable plug disclosed herein.

The dislodgeable plug 530 may be designed to work in conjunction with a stopper-piston. In such cases, the entire stopper-piston may be slidably positioned in a sealing relationship with the neck of the vessel. The stopper-piston may be slidable from a first position to a second position. In the first position, the entire stopper-piston may be adjacent the proximal end of the vessel. In the second position, the entire stopper-piston may be closer to the distal end of the vessel than to the proximal end of the vessel.

The mixing vial system may be used in conjunction with a syringe system, such as a cartridge-style syringe. The syringe system may include a syringe with an axially movable plunger having a distal end defining a shoulder. A harpoon may extend axially from the distal end of the plunger. The harpoon may be static with respect to the plunger. The shoulder of the plunger may have a larger diameter than a harpoon minimum diameter.

When the mixing vial system is disposed in the cartridge-style syringe and the plunger is axially moved, the harpoon may penetrate the closed proximal end of the stopper-piston. The harpoon may then contact the dislodgeable plug 530 and fully dislodge the dislodgeable plug 530 prior to the shoulder of the plunger contacting the closed proximal end of the stopper-piston.

In some implementations, the plug 530 shown in FIGS. 19-21 can be used in a mixing vial that is not configured as a cartridge, similar to the mixing vials and stoppers describe with respect to FIGS. 3-4. In some implementations, the plug 530 shown in FIGS. 19-21 can be used in a mixing vial configured as a cartridge in which the stopper is a static stopper at the distal end of the mixing vial, similar to the mixing vials and stoppers describe with respect to FIG. 10.

The interaction between these elements allows for the separation of different substances within the mixing vial until the moment of use. When activated, the dislodgeable plug 530 is removed, allowing the separated substances to mix. The design of the dislodgeable plug 530 facilitates both secure positioning within the stopper and efficient removal when needed.

A number of example implementations are provided herein. However, it is understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device are disclosed herein, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

1. A syringe system, the system comprising:
a vessel, the vessel comprising a hollow interior chamber having a proximal end and a distal end, and a neck adjacent the proximal end, wherein the neck has a distal end and a proximal open end;
a stopper comprising a hollow interior chamber with an open distal end and a closed proximal end; and
a dislodgeable plug having a body and a radially outwardly extending flange, wherein the body has a longitudinal axis, a first axial end, and a second axial end opposite and spaced apart along the longitudinal axis from the first end, wherein the dislodgeable plug is disposed within and sealing the open distal end of the stopper, wherein the entire dislodgeable plug is fully dislodgeable as a single piece from the open distal end of the stopper, wherein the radially outwardly extending flange is disposed at the first axial end of the dislodgeable plug, wherein the dislodgeable plug has two static resting positions consisting of a first static resting position in which the plug rests on the first axial end and a second static resting position in which the plug rests on the second axial end.

2. The syringe system of claim 1, wherein the distal end of the vessel is a distal open end.

3. The syringe system of claim 1, wherein the stopper defines an inwardly facing annular groove that receives the radially outwardly extending flange of the dislodgeable plug.

4. The syringe system of claim 3, wherein the stopper defines a radially inwardly extending flange, wherein the radially inwardly extending flange defines the inwardly facing annular groove.

5. The syringe system of claim 1, wherein the interior chamber of the vessel comprises an anesthetic, and the interior chamber of the stopper comprises a buffering reagent.

6. The syringe system of claim 1, wherein the stopper comprises an elastomeric plastic or rubber.

7. The syringe system of claim 1, wherein the second axial end of the dislodgeable plug includes a strike plate, wherein the dislodgeable plug is disposed within the open distal end of the stopper such that the strike plate faces the hollow interior chamber of the stopper, the strike plate being configured to resist penetration by the harpoon when the harpoon is used to dislodge the dislodgeable plug.

8. The syringe system of claim 1, wherein a portion of the body of the dislodgeable plug between the second axial end and the radially outwardly extending flange is tapered from a largest cross-sectional area adjacent the radially outwardly extending flange to a smallest cross-sectional area adjacent the second axial end.

9. The syringe system of claim 1, wherein the first axial end defines a plug opening extending from the first axial end toward the second axial end.

10. The syringe system of claim 9, wherein side walls of the plug opening are tapered such that the plug opening has a larger diameter adjacent the first axial end than it does adjacent the second axial end.

11. The syringe system of claim 10, wherein the taper of the side walls of the plug opening includes a constant taper portion and a varying taper portion.

12. The syringe system of claim 11, wherein the varying taper portion is disposed adjacent the first axial end and the constant taper portion is disposed adjacent the second axial end.

13. The syringe system of claim 1, wherein the stopper comprises a stopper-piston, wherein the entire stopper-piston is slidably positioned in a sealing relationship with the neck.

14. The syringe system of claim 13, wherein the entire stopper-piston is slidable from a first position to a second position, wherein the entire stopper-piston is adjacent the proximal end of the vessel when in the first position, and wherein the entire stopper-piston is closer to the distal end of the vessel than to the proximal end of the vessel when in the second position.

15. A syringe system, the system comprising:
a syringe with an axially movable plunger having a distal end defining a shoulder, wherein a harpoon extends axially from the distal end of the plunger, wherein the shoulder has a larger diameter than a harpoon minimum diameter, wherein the harpoon is static with respect to the plunger; and
the syringe system of claim 13,
wherein, when the syringe system is disposed in the syringe and the plunger is axially moved, the harpoon penetrates the closed proximal end of the stopper-piston, contacts the dislodgeable plug, and fully dislodges the dislodgeable plug prior to the shoulder of the plunger contacting the closed proximal end of the stopper-piston.

16. A syringe system, the system comprising:
a vessel, the vessel comprising a hollow interior chamber having a proximal end and a distal end, and a neck adjacent the proximal end, wherein the neck has a distal end and a proximal open end;
a stopper comprising a hollow interior chamber with an open distal end and a closed proximal end; and
a dislodgeable plug having a body and a radially outwardly extending flange, wherein the body has a longitudinal axis, a first axial end, and a second axial end opposite and spaced apart along the longitudinal axis from the first end, wherein the dislodgeable plug is disposed within and sealing the open distal end of the stopper, wherein the entire dislodgeable plug is fully dislodgeable as a single piece from the open distal end of the stopper, wherein the radially outwardly extending flange is disposed at the first axial end of the dislodgeable plug, wherein the first axial end defines a plug opening extending from the first axial end toward the second axial end.

17. The syringe system of claim 16, wherein the distal end of the vessel is a distal open end.

18. The syringe system of claim 16, wherein the stopper defines an inwardly facing annular groove that receives the radially outwardly extending flange of the dislodgeable plug.

19. The syringe system of claim 18, wherein the stopper defines a radially inwardly extending flange, wherein the radially inwardly extending flange defines the inwardly facing annular groove.

20. The syringe system of claim 16, wherein the interior chamber of the vessel comprises an anesthetic, and the interior chamber of the stopper comprises a buffering reagent.

21. The syringe system of claim 16, wherein the stopper comprises an elastomeric plastic or rubber.

22. The syringe system of claim 16, wherein the second axial end of the dislodgeable plug includes a strike plate, wherein the dislodgeable plug is disposed within the open distal end of the stopper such that the strike plate faces the hollow interior chamber of the stopper, the strike plate being configured to resist penetration by the harpoon when the harpoon is used to dislodge the dislodgeable plug.

23. The syringe system of claim 16, wherein a portion of the body of the dislodgeable plug between the second axial end and the radially outwardly extending flange is tapered from a largest cross-sectional area adjacent the radially outwardly extending flange to a smallest cross-sectional area adjacent the second axial end.

24. The syringe system of claim 16, wherein side walls of the plug opening are tapered such that the plug opening has a larger diameter adjacent the first axial end than it does adjacent the second axial end.

25. The syringe system of claim 24, wherein the taper of the side walls of the plug opening includes a constant taper portion and a varying taper portion.

26. The syringe system of claim 25, wherein the varying taper portion is disposed adjacent the first axial end and the constant taper portion is disposed adjacent the second axial end.

27. The syringe system of claim 16, wherein the dislodgeable plug has two static resting positions consisting of a first static resting position in which the plug rests on the first axial end and a second static resting position in which the plug rests on the second axial end.

28. The syringe system of claim 16, wherein the stopper comprises a stopper-piston, wherein the entire stopper-piston is slidably positioned in a sealing relationship with the neck.

29. The syringe system of claim 28, wherein the entire stopper-piston is slidable from a first position to a second position, wherein the entire stopper-piston is adjacent the proximal end of the vessel when in the first position, and wherein the entire stopper-piston is closer to the distal end of the vessel than to the proximal end of the vessel when in the second position.

30. A syringe system, the system comprising:
a syringe with an axially movable plunger having a distal end defining a shoulder, wherein a harpoon extends axially from the distal end of the plunger, wherein the shoulder has a larger diameter than a harpoon minimum diameter, wherein the harpoon is static with respect to the plunger; and
the syringe system of claim 28,
wherein, when the syringe system is disposed in the syringe and the plunger is axially moved, the harpoon penetrates the closed proximal end of the stopper-piston, contacts the dislodgeable plug, and fully dislodges the dislodgeable plug prior to the shoulder of the plunger contacting the closed proximal end of the stopper-piston.

31.-70. (canceled)
